(19) United States
(12) Patent Application Publication
(10) Pub. No.: US 2026/0240152 A1
(43) Pub. Date: Aug. 20, 2026

(54) FUNGAL PATHOGEN GROWTH-INHIBITION PEPTIDES

(71) Applicant: Viosimos Agriculture LLC, Lincoln, NE (US)

(72) Inventors: Daniel Schachtman, Lincoln, NE (US); Gary Stacey, Columbia, MO (US); Dong Xu, Columbia, MO (US)

(21) Appl. No.: 19/444,385

(22) Filed: Jan. 9, 2026

Related U.S. Application Data
(60) Provisional application No. 63/743,826, filed on Jan. 10, 2025.
(60) Provisional application No. 63/752,106, filed on Jan. 31, 2025.

(51) Int. Cl. A01N 37/46; A01N 43/36; A01N 43/38; A01N 43/50; A01P 3/00; C07K 7/08

(52) U.S. Cl. CPC A01N 37/46; A01N 43/36; A01N 43/38; A01N 43/50; A01P 3/00; C07K 7/08

(57) ABSTRACT

Systems and methods for inhibition of fungal pathogen growth, such as fungal pathogen growth on agricultural products, are described. In a system embodiment, an antipathogenic composition includes, but is not limited to, a peptide having a nucleotide structure of at least one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and SEQ ID NO: 41.

### CROSS-REFERENCE TO RELATED APPLICATIONS

The present application claims the benefit of 35 U.S.C. § 119 (e) of U.S. Provisional Application Ser. No. 63/743,826, filed Jan. 10, 2025, and titled “FUNGAL PATHOGEN GROWTH-INHIBITION PEPTIDES” and of U.S. Provisional Application Ser. No. 63/752,106, filed Jan. 31, 2025, and titled “FUNGAL PATHOGEN GROWTH-INHIBITION PEPTIDES”. U.S. Provisional Application Ser. Nos. 63/743,826 and 63/752,106 are each incorporated by reference in its entirety.

### INCORPORATION OF SEQUENCE LISTING

The sequence listing contained in the file named “201960001USU1-Sequence-Listing-1-7-2026-ST26.xml”, which is 41,670 bytes in size (measured in MS-Windows®), contains 41 sequences, and which was created on Jan. 7, 2026, is contemporaneously filed with this specification by electronic submission (using the United States Patent Office EFS-Web filing system) and is incorporated herein by reference in its entirety.

### BACKGROUND

Fungal pathogens and the associated diseases caused thereby to crops worldwide represent a large and economically significant challenge. Fungal pathogens can cause loss of crops before and after harvest, thereby jeopardizing global food production and food security.

### SUMMARY

Systems and methods for inhibition of fungal pathogen growth, such as fungal pathogen growth on agricultural products, are described. In a system embodiment, an antipathogenic composition includes, but is not limited to, a peptide having a nucleotide structure of at least one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and SEQ ID NO: 41 as well as derivatives of each of these peptides in which a toxic amino acid (e.g., L-ethionine or fluorophenylalanine) is incorporated at either the N- or C-terminus.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### DRAWINGS

The Detailed Description is described with reference to the accompanying figures. In the figures, the use of the same reference numbers in different instances in the description and the figures may indicate similar or identical items.

FIG. 1A is a table of peptide compositions for the inhibition of growth of multiple fungal pathogens in accordance with example embodiments of the present disclosure.

FIG. 1B is a table of alanine-substituted peptide composition variants of the P18 peptide of FIG. 1A in accordance with example embodiments of the present disclosure.

FIG. 2A is a photograph of assay results of the inhibition of growth of fungal pathogens for peptide P18 of FIG. 1A on fungal species Pythium ultimum.

FIG. 2B is a photograph of assay results of the inhibition of growth of fungal pathogens for peptide P18 of FIG. 1A on fungal species Fusarium oxysporum.

FIG. 2C is a photograph of assay results of the inhibition of growth of fungal pathogens for peptide P18 of FIG. 1A on fungal species Botrytis cinerea.

FIG. 2D is a photograph of assay results of the inhibition of growth of fungal pathogens for peptide P18 of FIG. 1A on fungal species Verticillium dahliae.

FIG. 2E is a photograph of assay results of the inhibition of growth of fungal pathogens for peptide P18 of FIG. 1A on fungal species Pythium uncinulatum.

FIG. 2F is a photograph of assay results of the inhibition of growth of fungal pathogens for peptide P18 of FIG. 1A on fungal species Sclerotinia minor.

FIG. 3A is a photograph of assay results of the inhibition of growth of fungal pathogens for peptide P18 of FIG. 1A on fungal species Pythium ultimum present in water and in dimethyl sulfoxide (DMSO).

FIG. 3B is a photograph of assay results of the inhibition of growth of fungal pathogens for peptide P18 of FIG. 1A on fungal species Sclerotinia minor present in water and in dimethyl sulfoxide (DMSO).

FIG. 3C is a photograph of assay results of the inhibition of growth of fungal pathogens for peptide P18 of FIG. 1A on fungal species Verticillium dahliae present in water and in dimethyl sulfoxide (DMSO).

FIG. 4A is a photograph of assay results of the inhibition of growth of Pythium ultimum for lettuce plants treated with peptide P18 of FIG. 1A six weeks after inoculation.

FIG. 4B is a photograph of assay results of the inhibition of growth of Pythium ultimum for lettuce plants treated with water six weeks after inoculation without a corresponding peptide treatment.

FIG. 5A is a photograph of assay results of the inhibition of growth of Pythium ultimum for lettuce plants treated with peptide P18 of FIG. 1A eight weeks after inoculation.

FIG. 5B is a photograph of assay results of the inhibition of growth of Pythium ultimum for lettuce plants treated with water eight weeks after inoculation without a corresponding peptide treatment.

FIGS. 6A and 6B are photographs of assay results of the inhibition of growth of Verticillium dahliae with respect to roots of lettuce plants treated with peptide P18 of FIG. 1A shown with lettuce root cell samples six weeks after inoculation.

FIGS. 6C and 6D are photographs of assay results of the inhibition of growth of Verticillium dahliae with respect to roots of lettuce plants treated with water shown with lettuce root cell samples six weeks after inoculation without a corresponding peptide treatment.

FIG. 7 is a table of peptide compositions tested for the inhibition of yeast growth of multiple yeast strains in accordance with example embodiments of the present disclosure.

FIG. 8 is a photograph of assay results of the inhibition of growth of yeast for each of the peptide compositions of FIG. 7 on yeast species Candida albicans in accordance with example embodiments of the present disclosure.

FIG. 9 is a photograph of assay results of the inhibition of growth of yeast for each of the peptide compositions of FIG. 7 on yeast species Saccharomyces cerevisiae in accordance with example embodiments of the present disclosure.

FIG. 10 is a photograph of assay results of the inhibition of growth of yeast for each of the peptide compositions of FIG. 7 on yeast species Pichia pastoris in accordance with example embodiments of the present disclosure.

FIG. 11 is a table of peptide compositions tested for the inhibition of growth of multiple fungal species in accordance with example embodiments of the present disclosure.

FIG. 12 is a chart illustrating stand count over time for minotaur lettuce cultivars planted in a field in the presence of Fusarium oxysoporum f.sp. lactucae, with the lettuce either treated with water, with peptide P18-A4, or with peptide P11-A9 in accordance with example embodiments of the present disclosure.

FIG. 13A is a photograph of assay results of the inhibition of growth of fungal pathogens for peptide P18 of FIG. 11 at various concentrations on fungal species Calonectria ilicicola in accordance with example embodiments of the present disclosure.

FIG. 13B is a photograph of assay results of the inhibition of growth of fungal pathogens for peptide P18-A4 of FIG. 11 at various concentrations on fungal species Calonectria ilicicola in accordance with example embodiments of the present disclosure.

FIG. 13C is a photograph of assay results of the inhibition of growth of fungal pathogens for peptide P11-A9 of FIG. 11 at various concentrations on fungal species Calonectria ilicicola in accordance with example embodiments of the present disclosure.

### DETAILED DESCRIPTION

### Overview

Fungal diseases on crops caused by fungal pathogen proliferation can result in crop damage before and after harvest. For instance, an estimated 10 to 23 percent of crops are lost pre-harvest and an estimated 10 to 20 percent of crops are lost post-harvest due to fungal diseases. Current fungal treatments, such as chemical fungicides and fumigation, have been unable to significantly stem the losses. Fumigation in general has not been shown to be an economically viable treatment method for long-term fungal disease prevention. Moreover, for certain fungal pathogens that infect lettuce and other plant roots, there are currently no fungicides available to suitably control these diseases. Further, the use of genetically uniform crops can hasten the growth of new fungal variants, including fungicide-resistant strains.

Accordingly, the present disclosure is directed, at least in part, to systems and methods for inhibition of fungal pathogen growth, such as fungal pathogen growth on agricultural products, utilizing peptides or peptide compositions that prevent plants from becoming infected by fungal pathogens under agricultural field conditions. The peptides can control fungal pathogens or other plant diseases for a variety of agricultural products including, but not limited to, leafy greens (e.g., lettuce) and other plants (e.g., strawberries, tomatoes, wheat, corn, soybeans, grapevines, cucumbers, etc.).

In aspects, the peptide compositions described herein can be applied to targets using any suitable application technique. For example, the peptide compositions can be applied using a liquid suspension to soils, where the peptide compositions are present in the liquid suspension as individual active ingredients or combined with other compounds or biomolecules. Other application techniques include, but are not limited to, injecting via irrigation water, mixing with dry fertilizer for introduction to soil, and including as a seed treatment prior to planting. For above-ground fungal pathogens, these compositions could be sprayed directly to the crop. Alternatively or additionally, the peptide compositions described herein can be engineered to be expressed in plants for transgenic plant protection against fungal disease.

In aspects, the peptide compositions described herein can be included in agricultural and medical applications as a therapeutic agent for fungal infections with the peptide composition formulated as a topical application, formulated for intravenous injection, formulated as or with a medicament for oral administration, formulated for transgenic expression in plants, or the like.

### Example Implementations

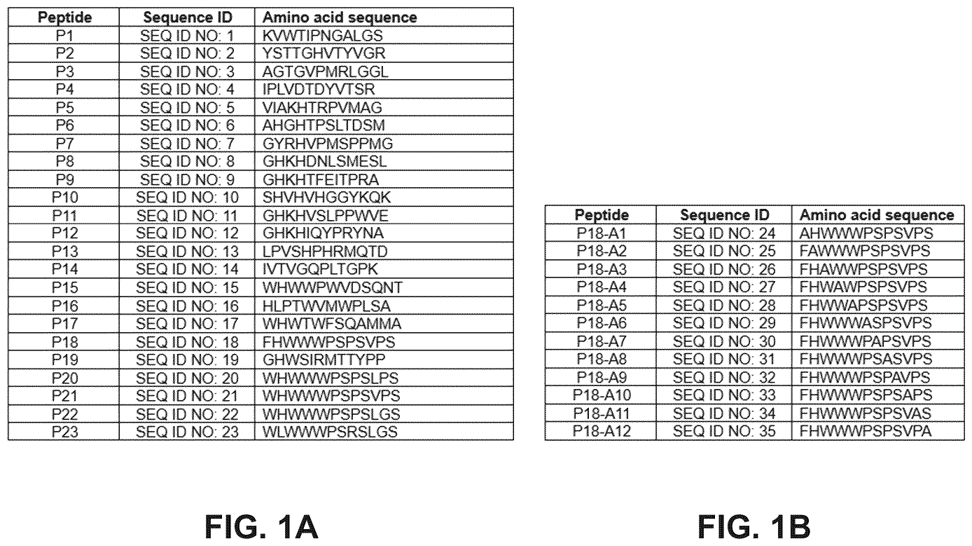

Peptides for the inhibition of growth of multiple fungal pathogens are provided in FIGS. 1A, 1B, 7, and 11, where the peptides provided in FIG. 1B are alanine-substituted variants of the P18 peptide of FIG. 1A. The peptides, which can be provided individually as an antipathogenic polypeptide or combined with multiple peptides, provide peptide compositions that facilitate inhibition of growth of multiple fungal pathogens simultaneously. For instance, the peptides have demonstrated fungal growth-inhibition properties against one or more fungal species of Pythium ultimum, Fusarium oxysporum, Botrytis cinerea, Verticillium dahliae, Pythium uncinulatum, and Sclerotinia minor, as well as selected yeast species (i.e., Saccharomyces cerevisiae, Candida albicans, and Pichia pastoris). The peptide compositions can be applied to soil configured to support an agricultural product according to a variety of application techniques. For example, the peptide compositions can be included in a liquid suspension for direct or indirect application to soil used to grow agricultural products. Alternatively or additionally, the peptide compositions can be included in an irrigation water mixture to be dispensed onto soil used to grow agricultural products. Alternatively or additionally, the peptide compositions can be included in a fertilized mixture to be added to soil used to grow agricultural products. Alternatively or additionally, the peptide compositions can be included as a coating to seeds to protect plants or to protect fruits or vegetables from post-harvest fungal infection.

In embodiments, the peptide composition can include an amino acid sequence of FHWWXPSYSVPS, where X can be any amino acid and Y can be any amino acid. For example, in implementations, at least one of X and Y is alanine. In implementations, X can be any amino acid and Y is proline. In implementations, Y can be any amino acid and X is tryptophan.

### Fungal Pathogen Growth-Inhibition Peptide Experimental Examples

Various peptide compositions of FIG. 1A (SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23) were analyzed to determine the antifungal effects of each peptide on fungal species of Pythium ultimum, Pythium uncinulatum, Fusarium oxysporum, Botrytis cinerea, Sclerotinia minor, and Verticillium dahliae according to the following methodology.

### Fungal Growth, Media and Fungal Spore Collection

Fungal species of Pythium ultimum, Pythium uncinulatum, Fusarium oxysporum, Botrytis cinerea, Sclerotinia minor, and Verticillium dahliae were used in the study. Pythium ultimum and Pythium uncinulatum were grown on V8 agar plates. Fusarium oxysporum, Botrytis cinerea, Sclerotinia minor, and Verticillium dahliae were grown on potato dextrose agar (PDA) plates. All fungal species were grown at room temperature in the dark. The spores were washed off plates using one quarter (¼) strength potato dextrose broth from 2-week-old fungal plates and filtered through a 100 μm filter. The spores were collected into 50 mL conical centrifuge tubes. The spore concentration was determined using a hemocytometer under a microscope. The spore suspension was diluted to various concentrations by adding one quarter (¼) strength potato dextrose broth for use.

### Peptides

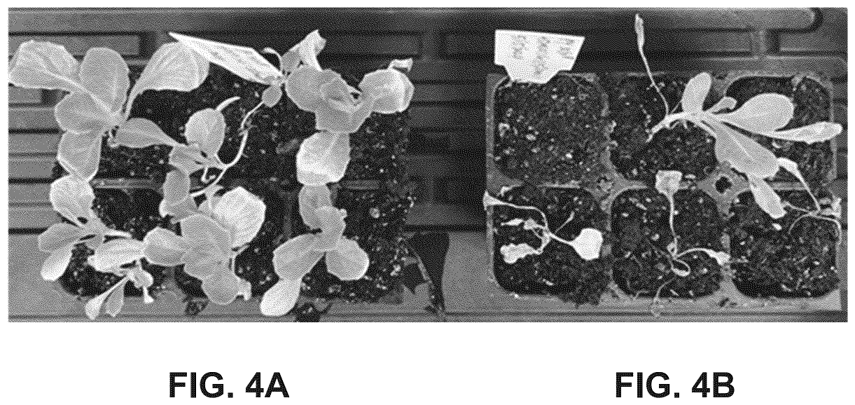

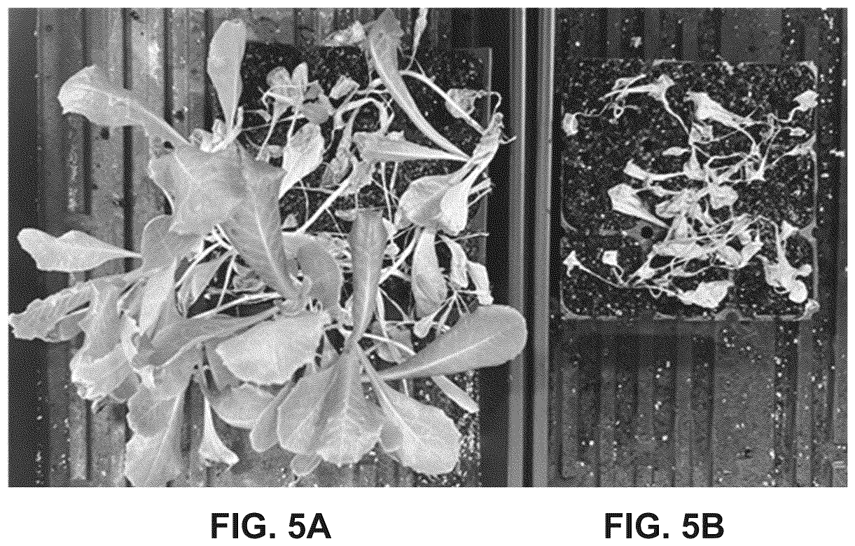

Various peptides having compositions corresponding those provided in FIG. 1A were synthesized and purified to at least 95% purity. The amount synthesized was from 2 to 5 mg for each peptide. All peptides were dissolved in dimethyl sulfoxide (DMSO) or dimethylformamide (DMF) based on the amino acid content of each peptide to make a 10 mM stock solution and then diluted to 1 mM solution using water. The peptides containing cysteine (Cys) or methionine (Met) (i.e., P3, P5, P6, P7, P8, P13, P16, P17, P19) were dissolved in DMF and the remainder of the peptides (i.e., P1, P2, P4, P9, P10, P11, P12, P14, P15, P18) were dissolved in DMSO. It was observed that the purity of peptide P18 after synthesis was over 95% without further purification, which can significantly reduce the cost of large-scale peptide synthesis. Accordingly, a larger amount (25 gram) of P18 was synthesized for plant protection and other tests (e.g., for testing corresponding to FIGS. 4A through 5B, described herein below).

For initial observations of peptide inhibition of fungal growth, 1 μL of fungal spore suspension (1×10⁶ CFU/mL) for each of the fungal species of Pythium ultimum, Fusarium oxysporum, Botrytis cinerea, and Verticillium dahliae was added to 10 μL of 1 mM of peptide solution, mixed, and maintained at room temperature for 30 to 60 minutes. As a control, 1 μL of spores was also similarly treated with 10 μL of water, 10% of DMSO, or 10% DMF depending on the amino acid composition of the individual peptides as described above. These mixtures were applied to PDA or V8 agar medium plates in divided Petri dishes to provide inoculated media. The inoculated media were cultured at room temperature in the dark for 3 to 7 days. The growth of fungus was visually observed.

Results of the assays for peptide inhibition of fungal growth for the inoculated media are shown in Table 1, where levels of inhibition were ranked from 0 to 5+, where 0 indicated that no antifungal effect was observed and where 5+ indicated that no fungal growth was observed 3-5 days after incubation.

TABLE 1

| | Pythium | Fusarium | Botrytis | Sclerotinia |
|---|---|---|---|---|
| Peptide | ultimum | oxysporum | cinerea | minor |
| P1 | 1+ | 1+ | 0 | 0 |
| P2 | 3+ | 3+ | 4+ | 1+ |
| P3 | 2+ | 2+ | 0 | 1+ |
| P4 | 1+ | 2+ | 0 | 0 |
| P5 | 2+ | 1+ | 3+ | 1+ |
| P6 | 1+ | 1+ | 1+ | 5+ |
| P7 | 1+ | 2+ | 3+ | 5+ |
| P8 | 5+ | 3+ | 4+ | 1+ |
| P9 | 3+ | 3+ | 5+ | 1+ |
| P10 | 5+ | 4+ | 5+ | 1+ |
| P11 | 5+ | 5+ | 5+ | 5+ |
| P12 | 5+ | 4+ | 5+ | 1+ |
| P13 | 1+ | 1+ | 1+ | 1+ |
| P14 | 2+ | 2+ | 1+ | 1+ |
| P15 | 3+ | 4+ | 3+ | 3+ |
| P16 | 3+ | 4+ | 3+ | 2+ |
| P17 | 2+ | 3+ | 2+ | 1+ |
| P18 | 5+ | 5+ | 5+ | 5+ |
| P19 | 3+ | 4+ | 2+ | 1+ |

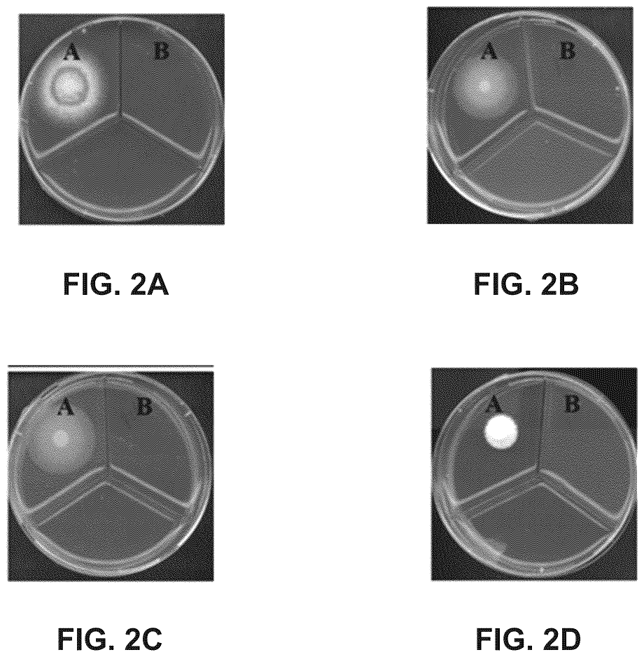

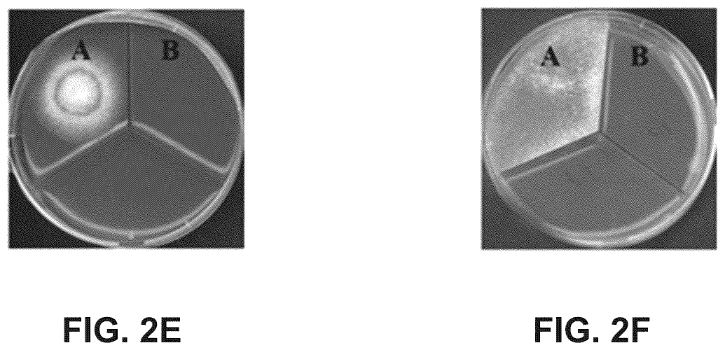

The assays revealed that all the peptides of FIG. 1A shown in Table 1 inhibited growth of the four tested fungal species, with varying levels of effectiveness. Peptides P18 and P11 exhibited the highest levels of inhibition of fungal growth during the assays, followed by P10 and P12. Example results of efficacy of peptide P18 on the inhibition of growth of fungal pathogens are shown in FIGS. 2A through 2F, where FIGS. 2A through 2D show results of the assays involving Pythium ultimum, Fusarium oxysporum, Botrytis cinerea, and Verticillium dahliae, respectively, and where FIGS. 2E and 2F show results of assays involving Pythium uncinulatum and Sclerotinia minor, respectively. Plate sections labeled as A correspond to the control section that includes the fungal spores treated with 10% DMSO without a corresponding peptide treatment, whereas plate sections labeled as B correspond to the fungal spores treated with 1 mM peptide P18 in 10% DMSO.

### Peptide P18 Functionality in Water and DMSO

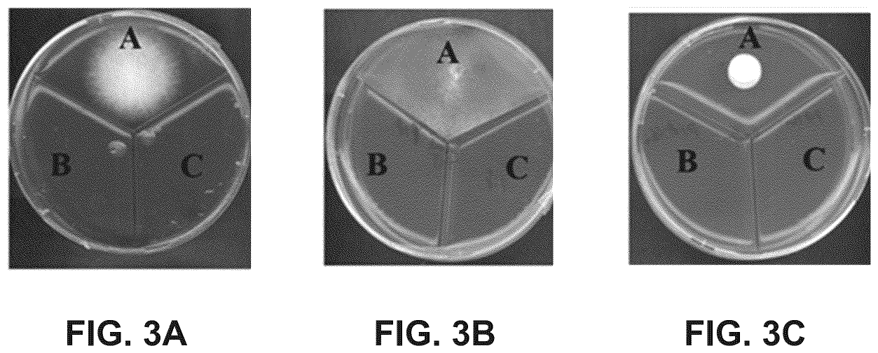

The effects of peptide P18 on inhibition of growth of fungal pathogens in each of water and DMSO were tested. Three fungal species, Pythium ultimum, Sclerotinia minor, and Verticillium dahliae, were tested to compare the effects of peptide P18 in water and in DMSO. Three sample mixtures were applied to individual medium plates in divided Petri dishes to provide inoculated media, where a first sample included the respective fungal spores treated with water without a corresponding peptide treatment, a second sample included the respective fungal spores treated with 1 mM peptide P18 in water, and a third sample included the respective fungal spores treated with 1 mM peptide P18 in 10% DMSO. FIGS. 3A through 3C show results of the assays involving Pythium ultimum, Sclerotinia minor, and Verticillium dahliae, respectively, where plate sections labeled as A correspond to the control section that include the first sample (i.e., fungal spores treated with water), plate sections labeled as B correspond to the water section that include the second sample (i.e., fungal spores treated with peptide P18 in water), and plate sections labeled as C correspond to the DMSO section that include the third sample (fungal spores treated with peptide P18 in DMSO). The results demonstrated that the peptide P18 inhibitory effect is strongly active when dissolved in water, which can be suitable for applications to soil or above-ground plant parts in agricultural fields.

Plant Protection with P18 Peptides

The effects of peptide P18 on growth of fungal pathogens in each of water and DMSO as applied to plants were tested. Lettuce plants in soil contained in small pots were treated with fungal spores provided in water as a control or with 1 mM peptide P18. The fungal spores were Pythium ultimum spores in an amount of 1×10⁸ CFU/mL. The soil around the lettuce plants was inoculated with the control treatment or the peptide P18 treatment and the lettuce plants were observed at time durations of six weeks after inoculation and eight weeks after inoculation. Results are shown in FIGS. 4A through 5B, where FIG. 4A corresponds to lettuce plants treated with the peptide P18 treatment six weeks after inoculation, FIG. 4B corresponds to lettuce plants treated with the control treatment six weeks after inoculation, FIG. 5A corresponds to lettuce plants treated with the peptide P18 treatment eight weeks after inoculation, and FIG. 5B corresponds to lettuce plants treated with the control treatment eight weeks after inoculation.

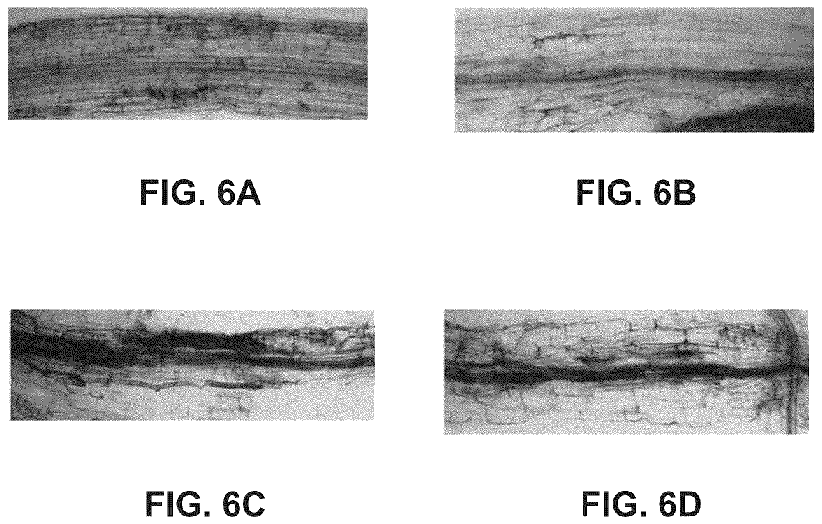

The effects of peptide P18 on infection of fungal pathogens on plant roots were tested. Two-week old lettuce plants were first inoculated with peptide P18-treated Verticillium dahliae spores (5×10⁷ CFU/mL) or water-treated Verticillium dahliae spores (5×10⁷ CFU/mL) and subsequently inoculated with the respective treatments again at one week after the first inoculation. Lettuce roots from each of the plants with peptide P18-treated Verticillium dahliae spores and with water-treated Verticillium dahliae spores were sampled six weeks after the first inoculation and were stained with trypan blue. Verticillium dahliae infects the vascular tissue of plant roots and shows a blue color after trypan blue staining. Results of the assays are shown in FIGS. 6A through 6D, where FIGS. 6A and 6B correspond to stained roots of the peptide P18-treated samples and FIGS. 6C and 6D correspond to stained roots of the water-treated samples. The staining results revealed that the peptide P18-treated spores significantly reduced infection relative to water-treated spores in the infected root tissues, particularly observed in the vascular tissues.

### Peptide P18 Substitutions and Effects on Fungal Growth

Peptide P18 and variants of peptide P18 having single nucleic acids substituted with alanine amino acid (e.g., shown in FIG. 1B as SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35) were tested to observe the effects on growth of Pythium ultimum, Fusarium oxysporum, and Verticillium dahliae spores with experimental conditions similar to those provided above with respect to the assays for peptide inhibition of fungal growth associated with the results in Table 1. The assay results for peptide inhibition of fungal growth for Peptide P18 and the variants of peptide P18 are provided in Table 2, where levels of inhibition were ranked from 0 to 5+, where 0 indicated that no antifungal effect was observed and where 5+ indicated complete inhibition of fungal growth.

TABLE 2

| | Amino | Pythium | Fusarium | Verticillium |
|---|---|---|---|---|
| Peptide | acid sequence | ultimum | oxysporum | dahliae |
| P18 | FHWWWPSPSVPS | 5+ | 5+ | 5+ |
| P18-A1 | AHWWWPSPSVPS | 4+ | 3+ | 3+ |
| P18-A2 | FAWWWPSPSVPS | 3+ | 2+ | 2+ |
| P18-A3 | FHAWWPSPSVPS | 3+ | 2+ | 3+ |
| P18-A4 | FHWAWPSPSVPS | 4+ | 4+ | 3+ |
| P18-A5 | FHWWAPSPSVPS | 5+ | 5+ | 5+ |
| P18-A6 | FHWWWASPSVPS | 4+ | 5+ | 5+ |
| P18-A7 | FHWWWPAPSVPS | 4+ | 5+ | 5+ |
| P18-A8 | FHWWWPSASVPS | 5+ | 5+ | 5+ |
| P18-A9 | FHWWWPSPAVPS | 3+ | 3+ | 3+ |
| P18-A10 | FHWWWPSPSAPS | 3+ | 4+ | 5+ |
| P18-A11 | FHWWWPSPSVAS | 3+ | 3+ | 5+ |
| P18-A12 | FHWWWPSPSVPA | 4+ | 4+ | 4+ |

Analysis of the results of the assays for Peptide P18 and the variants of peptide P18 indicated that 10 of the 12 amino acids provide substantive effects to inhibition of fungal growth, where the fifth and eighth amino acids from the N-terminal can be replaceable without substantive effects to inhibition of fungal growth, particularly when substituted with alanine.

Yeast Growth Inhibition with Peptides

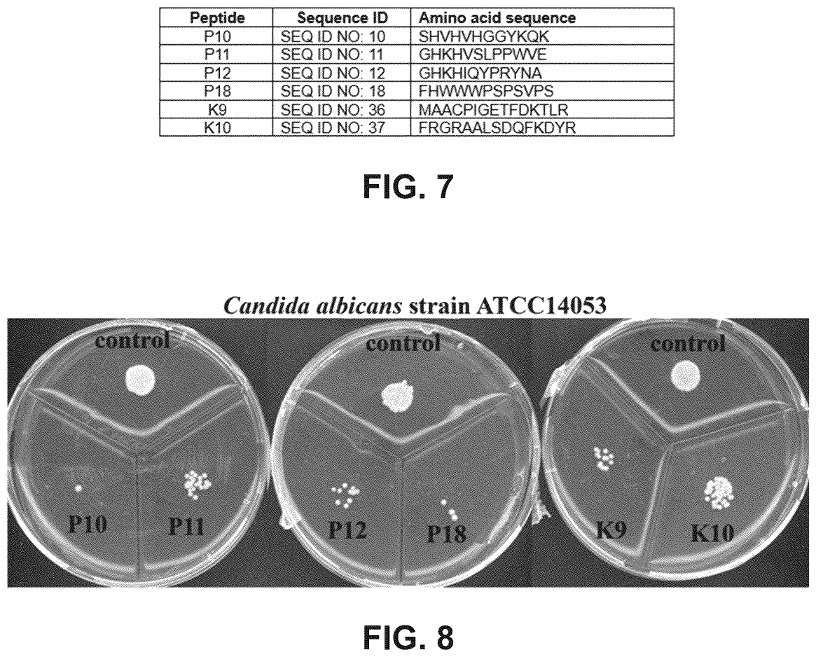

Peptides having compositions corresponding to those provided in FIG. 7 (SEQ ID NO: 10, SEQ ID NO: 11, SEQ ID NO: 12, SEQ ID NO: 18, SEQ ID NO: 36, SEQ ID NO: 37) were tested to observe the effects on growth of three yeast strains including Candida albicans strain ATCC14053, Saccharomyces cerevisiae strain PJ69-4a, and Pichia pastoris strain X-33. The yeast strains were grown in Yeast Peptone Dextrose (YPD) medium at 30° C.

Peptides P10, P11, P12 and P18 were selected by phage display analysis against fungal spores, whereas K9 and K10 were selected as target-specific peptides to bind to chitin synthases. The peptides were synthesized in an amount from about 2 mg to about 5 mg for each peptide and purified to at least 95% purity. The peptides were dissolved in dimethyl sulfoxide (DMSO) or dimethylformamide (DMF) based on the amino acid content of each peptide to make a 10 mM stock solution and then diluted to 1 mM solution using water. The peptides containing cysteine (Cys) or methionine (Met) (i.e., K9) were dissolved in DMF and the remainder of the peptides were dissolved in DMSO (i.e., P10, P11, P12, P18, K10). It was observed that the purity of peptide P18 after synthesis was over 95% without further purification, which can significantly reduce the cost of large-scale peptide synthesis.

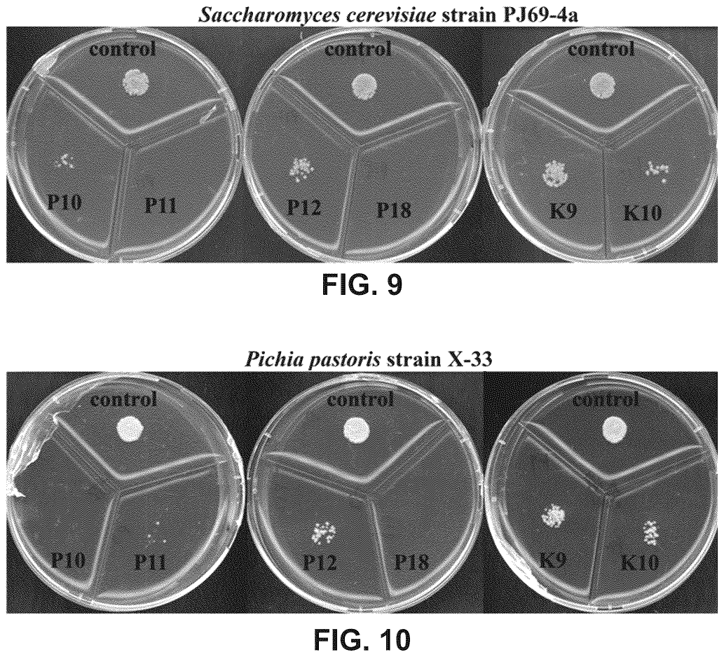

For the peptide inhibition of yeast growth assays, 1 μL of yeast culture (1×10⁷ CFU/mL) of each yeast strain (i.e., Candida albicans strain ATCC14053, Saccharomyces cerevisiae strain PJ69-4a, and Pichia pastoris strain X-33) was individually added to separate 10 μL volumes of 1 mM of peptide solution, mixed, and kept at room temperature for 60 minutes. As a control, 1 μL of yeast culture was also similarly treated with 10 μL of 10% of DMSO or 10% DMF depending on the amino acid composition of individual peptides, as described above. The mixtures were applied on YPD agar in divided Petri dishes. The inoculated media were cultured at 30° C. in the dark for 3 days, with daily visual observation beginning one day from inoculation. The growth of yeast was visually observed and pictures corresponding to those in FIGS. 8A through 10C were taken using a scanner.

Results of the assays for peptide inhibition of yeast growth for the inoculated media are shown in Tables 3 through 5, where Table 3 corresponds to assay results for Candida albicans strain ATCC14053, Table 4 corresponds to assay results for Saccharomyces cerevisiae strain PJ69-4a, and Table 5 corresponds to assay results for Pichia pastoris strain X-33, where levels of inhibition were ranked from 0 to 5+, where 0 indicated that no antifungal effect was observed and where 5+ indicated that no fungal growth was observed for each daily observation taken 1 to 3 days after incubation.

TABLE 3

| | ATCC14053 | ATCC14053 | ATCC14053 |
|---|---|---|---|
| Peptide | 1 day | 2 days | 3 days |
| P10 | 5+ | 4+ | 4+ |
| P11 | 5+ | 3+ | 5+ |
| P12 | 5+ | 3+ | 3+ |
| P18 | 5+ | 4+ | 4+ |
| K9 | 5+ | 4+ | 3+ |
| K10 | 4+ | 3+ | 2+ |

TABLE 4

| | PJ69-4a | PJ69-4a | PJ69-4a |
|---|---|---|---|
| Peptide | 1 day | 2 days | 3 days |
| P10 | 4+ | 4+ | 4+ |
| P11 | 5+ | 5+ | 5+ |
| P12 | 5+ | 3+ | 3+ |
| P18 | 5+ | 5+ | 5+ |
| K9 | 2+ | 2+ | 2+ |
| K10 | 4+ | 4+ | 4+ |

TABLE 5

| | X-33 | X-33 | X-33 |
|---|---|---|---|
| Peptide | 1 day | 2 days | 3 days |
| P10 | 5+ | 5+ | 4+ |
| P11 | 5+ | 4+ | 4+ |
| P12 | 5+ | 2+ | 2+ |
| P18 | 5+ | 5+ | 5+ |
| K9 | 5+ | 2+ | 2+ |
| K10 | 5+ | 3+ | 3+ |

The peptide inhibition of yeast growth assays revealed that each of the six tested peptides inhibited growth of the tested fungal species, with differing levels of inhibition between the peptides and between the particular fungal species. For instance, peptide P18 demonstrated the highest average inhibition of yeast growth for the three tested yeast strains, followed by peptide P10 and peptide P11. FIGS. 8 through 10 show results of the assays involving Candida albicans strain ATCC14053, Saccharomyces cerevisiae strain PJ69-4a, and Pichia pastoris strain X-33, respectively, two days after inoculation. The tray sections in FIGS. 8 through 10 which are labeled “control” include the yeast culture without exposure to any of the test peptides, whereas the tray sections including the peptide number (e.g., P10, P11, P12, P18, K9, K10) include the respective yeast culture with the corresponding peptide, with the control and peptide preparations described herein above.

Development of Enhanced Peptides with Lower Minimum Effective Concentrations

Peptide P18-04 (SHWWWHSGSKSS, SEQ ID NO: 41) was discovered through optimization of the original peptide P18 (FHWWWPSPSVPS) using plate assay data of other similar sequences to peptide P18. The anti-fungal efficiency was increased 10-fold for Fusariuim oxysporum, approximately 10-fold for Verticillium dahliae, approximately 2-fold for Botrytis cinerea, and approximately 10-fold for Pythium ultimum as compared to the original peptide P18. More effective plant protection from the fungal pathogen was also found in soil based experiments in growth chambers using the optimized peptide P18-04.

Similarly, peptide P11-A9 (GHKHTSLPPWVA, SEQ ID NO:38) was discovered through optimization of the original peptide P11 (GHKHVSLPPWVE) using plate assay data of other similar sequences to peptide P11. The anti-fungal efficiency increased was 2-fold for Fusariuim oxysporum, 4-fold for Verticillium dahliae, no observed improvement for Botrytis cinerea, and approximately 2-fold for Pythium ultimum.

Field Trial for Fungal Growth Inhibition with P11 and P18 Peptides

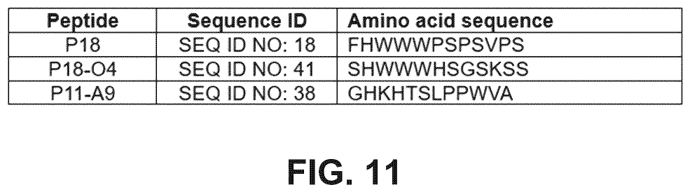

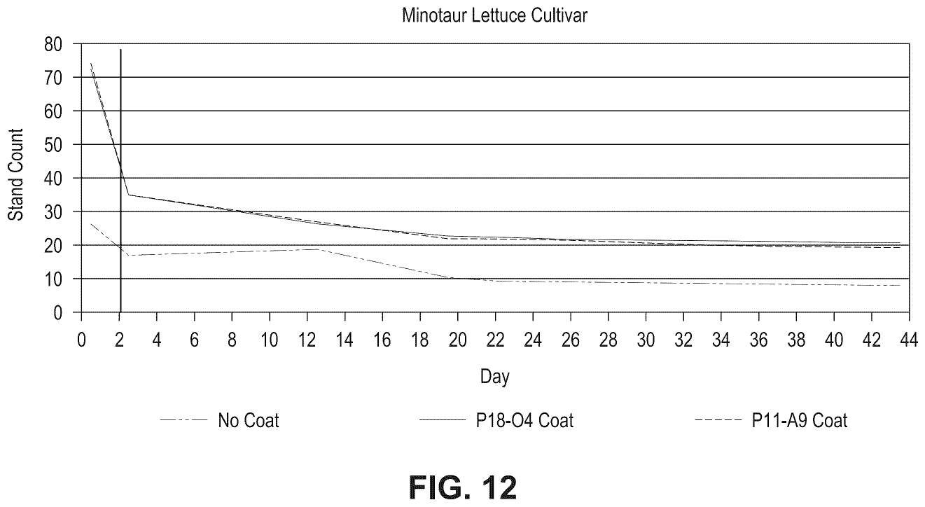

Peptides having compositions corresponding to those provided in FIG. 11 (SEQ ID NO: 18, SEQ ID NO: 41, and SEQ ID NO: 38) were tested to observe the effects on growth of lettuce cultivar Minotaur in the presence of fungal strain Fusarium oxysoporum f.sp. lactucae. A field trial was conducted in Yuma, Arizona in which the lettuce cultivar Minotaur was planted into four replicate plots in a field heavily infested with Fusarium oxysoporum f.sp. lactucae. Lettuce seed was initially treated with peptide P18-04 or with peptide P11-A9 and the seeds were planted and then thinned thirteen days later. The thinning eliminates some germinated plants to create space for the lettuce heads to fully develop. The soil where the lettuce was growing was treated with water, with peptide P18-04, or with peptide P11-A9 throughout the trial, with a first treatment the day after planting, a second treatment twelve days after planting, and a third treatment twenty-four days after planting. Results of the field trial are shown in FIG. 12, where the results indicated that after thinning, the peptide treatments increased the number of viable lettuce plants in each of the four replicate plots as compared to lettuce treated with water and no corresponding peptide treatment.

### Concentration of Peptides on Fungal Growth Inhibition

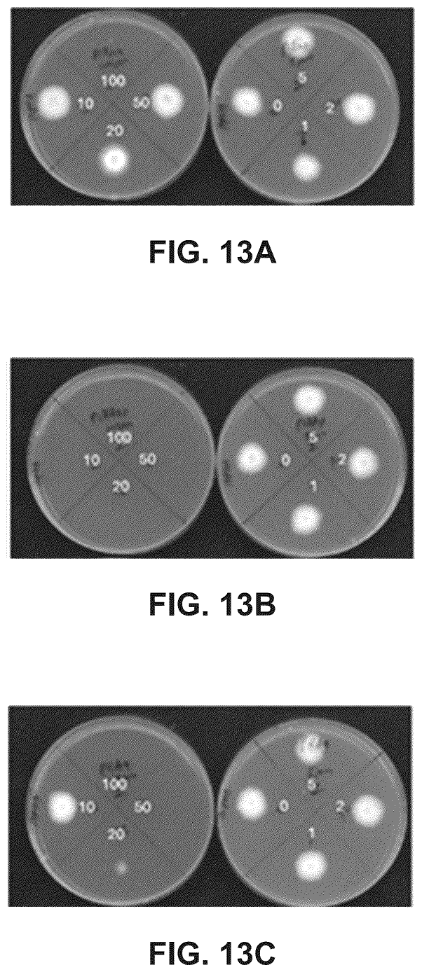

Peptides having compositions corresponding to those provided in FIG. 11 were tested with various concentrations to observe the effects on growth of fungal strain Calonectria ilicicola (red crown rot fungus), an emerging threat to soybean crops. Each peptide was applied to the fungal strain with a control (0 μM) and at concentrations of 1 μM, 2 μM, 5 μM, 10 μM, 20 μM, 50 μM, and 100 μM. Results of the assays are shown in FIGS. 13A through 13C, where FIG. 13A shows assay results for treatment with peptide P18, FIG. 13B shows assay results for treatment with peptide P18-A4, and FIG. 13C shows assay results for treatment with peptide P11-A9. The assay results show that peptide P18 inhibited the growth of fungal strain Calonectria ilicicola at a concentration of 100 μM and that peptides P18-A4 and P11-A9 inhibited the growth of fungal strain Calonectria ilicicola at lower concentrations (e.g., 10 UM and 20 μM, respectively).

### Peptide Composition Type and Stability in Soil

Soil conditions can affect the stability of peptides applied to agricultural soil. For instance, soil environments contain diverse microorganisms, their secreted proteases, and other chemical factors that can compromise peptide stability. The stability of peptide P18A4 in agricultural soil was tested by synthesizing an influenza hemagglutinin (HA) tagged version of P18A4 (HA-P18A4) and incubating the tagged peptide in agricultural soil. Initial assays indicated that HA-P18A4 degraded within a few days, as evidenced by the loss of signal detected with an anti-HA antibody. Without wishing to be bound to any particular mechanism, it is believed that the degradation could have occurred within the HA tag, the P18A4 sequence, or both.

D-peptides have shown resistance to proteolytic degradation and can possess longer half-lives than L-peptide counterparts. For instance, D-peptides are composed entirely of D-amino acids, which are mirror images of their corresponding L-amino acids. An all-D-amino acid version of P1804 (i.e., a D-peptide, P1804D, SEQ ID NO: 39) was synthesized and evaluated for biological activity. D-peptide P1804D inhibited fungal pathogens as effectively as the L-peptide version (i.e., peptide P1804). Based on the D-peptide composition, it is expected that peptide P1804D will have longer stability in agricultural soil conditions as compared to the L-peptide version (i.e., peptide P1804).

In embodiments, the peptide composition can include an amino acid sequence of FHWWXPSYSVPS (i.e., SEQ ID NO: 40), where X can be any amino acid and Y can be any amino acid. For example, in implementations, at least one of X and Y is alanine. In implementations, X can be any amino acid and Y is proline. In implementations, Y can be any amino acid and X is tryptophan.

The nucleic acids and polypeptides of the present disclosure find use in methods for inducing pathogen resistance in a plant. Accordingly, the compositions and methods disclosed herein are useful in protecting plants against fungal pathogens, bacteria, viruses, and the like. “Pathogen resistance” or “disease resistance” can mean that the plant avoids the disease symptoms that are the outcome of plant-pathogen interactions. That is, pathogens are prevented from causing plant diseases and the associated disease symptoms, or alternatively, the disease symptoms caused by the pathogen are minimized or lessened, such as, for example, the reduction of stress and associated yield loss. One of skill in the art will appreciate that the compositions and methods disclosed herein can be used with other compositions and methods available in the art for protecting plants from pathogen attack.

“Antipathogenic compositions” or “antipathogenic polypeptides” can mean that the compositions of the present disclosure have antipathogenic activity and thus are capable of suppressing, controlling, and/or killing the invading pathogenic organism. An antipathogenic polypeptide of the present disclosure will reduce the disease symptoms resulting from pathogen challenge by at least about 5% to about 50%, at least about 10% to about 60%, at least about 30% to about 70%, at least about 40% to about 80%, or at least about 50% to about 90% or greater. Hence, the methods of the present disclosure can be utilized to protect plants from disease, particularly those diseases that are caused by plant pathogens. In particular embodiments, the antipathogenic activity exhibited by the polypeptides of the present disclosure is antifungal activity. As used herein, “antifungal activity” can refer to the ability to suppress, control, and/or kill the invading fungal pathogen. Likewise, “fungal resistance” refers to enhanced tolerance to a fungal pathogen when compared to that of an untreated or wild type plant. Resistance may vary from a slight increase in tolerance to the effects of the fungal pathogen (e.g., partial inhibition) to total resistance such that the plant is unaffected by the presence of the fungal pathogen. An increased level of resistance against a particular fungal pathogen or against a wider spectrum of fungal pathogens may both constitute antifungal activity or improved fungal resistance.

As used herein, “nucleic acid” includes reference to a deoxyribonucleotide or ribonucleotide polymer in either single- or double-stranded form, and unless otherwise limited, encompasses known analogues (e.g., peptide nucleic acids) having the essential nature of natural nucleotides in that they hybridize to single-stranded nucleic acids in a manner similar to naturally occurring nucleotides, and residues thereof.

The terms “polypeptide,” “peptide,” and “protein” can be used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers. Polypeptides of the present disclosure can be produced either from a nucleic acid disclosed herein, or by the use of standard molecular biology techniques. For example, a truncated protein of the present disclosure can be produced by expression of a recombinant nucleic acid of the present disclosure in an appropriate host cell, or alternatively by a combination of ex vivo procedures, such as protease digestion and purification.

It will be appreciated that the peptides described herein may include amino acids having any combination of L-amino acids and D-amino acids. It will be appreciated that the peptides described herein may include one or more of N-terminal modification and C-terminal modification. Such modifications can include, but are not limited to, N-terminal acetylation and C-terminal amidation. For example, the peptides can include N-terminal modification with hydrogen, an acetyl group, or a propionyl group. Additionally or alternatively, the peptides can include, for example, C-terminal modification with —OH, an amide group, a methylamide group, or an ethylamide group. In implementations, N-terminal and/or C-terminal modification can include a toxic amino acid (e.g., L-ethionine or fluorophenylalanine) incorporated at either the N- or C-terminus.

In implementations, the peptides described herein may be provided as a peptide composition having one or more agriculturally-acceptable carriers in combination with one or more of the peptides described herein. As used herein, the term “agriculturally-acceptable” in reference to a carrier, material, ingredient, or substance of a peptide composition comprising a one or more of the peptides described herein refers to a carrier, material, ingredient, or substance that (i) is compatible with other ingredients of the peptide composition at least for the purpose in which the peptide composition will be used, (ii) can be included in the peptide composition to effectively and viably deliver the one or more peptides to a plant, plant part, plant seed, or plant growth medium (e.g., soil), (iii) is not normally associated with the one or more peptides in nature (at least in the form in which it will be used), and (iv) is not deleterious to a plant, plant part, or plant seed to which the peptide composition will be applied (at least in the manner and amount in which it will be applied to, or associated with, the plant, plant part, or plant seed).

A carrier includes any substance or material that may be used and/or combined with a microbial strain or isolate to improve the delivery or effectiveness of the microbial strain or isolate to a plant, plant part or plant seed. An agriculturally acceptable carrier may include a soil-compatible carrier, a seed-compatible carrier, and/or a foliar-compatible carrier. As used herein, the term “soil-compatible carrier” refers to a material that can be added or applied to a soil without causing/having an unduly adverse effect on plant yield, soil structure, soil drainage, or the like. The term “seed-compatible carrier” refers to a material that can be added or applied to a seed without causing/having an unduly adverse effect on the seed, seed germination, the plant that grows from the seed, or the like. The term “foliar-compatible carrier” refers to a material that can be added or applied to an above ground portion of a plant or plant part without causing/having an unduly adverse effect on plant yield, plant health, or the like. Selection of appropriate carrier materials will depend on the intended application(s) and the peptide(s) present in the composition. The carrier material(s) may be selected and/or combined to provide a composition or formulation in the form of a liquid, gel, slurry, or solid. Compositions may comprise one or more liquid and/or gel carriers, and/or one or more aqueous and/or non-aqueous solvents. As used herein, the term “non-aqueous” may refer to a composition, solvent, or substance that includes no more than a trace amount of water (e.g., no more than 0.5% water by weight).

The peptide compositions provided herein may include one or more additional agriculturally-acceptable components, such as in combination with or as a replacement to, the agriculturally-acceptable carrier. For instance, the one or more additional agriculturally-acceptable components can include, but are not limited to, carriers configured to provide the peptide composition in solid form, in liquid form, in gel form, or the like, solvents, buffers, thickeners, stabilizers, polymers, dispersants, and the like.

In implementations, the peptides described herein may be provided as a peptide composition having one or more pharmaceutically-acceptable excipients including, but not limited to carriers, emulsifiers, coemulsifiers, permeation or penetration enhancers, solvents, co-solvents, emollients, antioxidants, preservatives, buffering agents, gelling or thickening agents, polymers, surfactants, pH modifiers, solubilizers, and the like. The pharmaceutically-acceptable excipients can facilitate distribution of the peptide composition to an individual (e.g., for inhibition of fungal pathogen growth within or on the individual). In implementations, the peptides described herein may be provided as a peptide composition having one or more pharmaceutically-acceptable carriers having organic or inorganic ingredients, natural or synthetic, with which an active ingredient is combined to facilitate application of the peptide composition.

### CONCLUSION

It will be appreciated that features described herein with respect to embodiments or implementations can be combined with any other feature or features described with respect to the same or alternative embodiments, unless context otherwise dictates, without departing from the scope of the present disclosure.

Although the subject matter has been described in language specific to structural features and/or process operations, it is to be understood that the subject matter defined in the appended claims is not necessarily limited to the specific features or acts described above. Rather, the specific features and acts described above are disclosed as example forms of implementing the claims.

1. A peptide composition for the inhibition of fungal pathogen growth, the peptide composition comprising a nucleotide structure of at least one of:
SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and SEQ ID NO: 41.

2. The peptide composition of claim 1, further comprising an agriculturally-acceptable carrier.

3. The peptide composition of claim 1, further comprising a pharmaceutically-acceptable carrier.

4. The peptide composition of claim 1, wherein the nucleotide structure comprises at least one of SEQ ID NO: 18, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, and SEQ ID NO: 41.

5. The peptide composition of claim 4, further comprising an agriculturally-acceptable carrier.

6. The peptide composition of claim 4, further comprising a pharmaceutically acceptable carrier.

7. The peptide composition of claim 1, wherein the nucleotide structure comprises SEQ ID NO: 27.

8. The peptide composition of claim 7, wherein at least one amino acid of the nucleotide structure is a D-amino acid.

9. The peptide composition of claim 7, wherein each amino acid of the nucleotide structure is a D-amino acid.

10. The peptide composition of claim 9, further comprising an agriculturally-acceptable carrier.

11. A method of treating an agricultural product to inhibit fungal pathogen growth, comprising:
introducing the peptide composition of claim 1 to at least one of soil configured to support an agricultural product or a seed as a seed coating.

12. The method of claim 11, wherein the peptide composition includes an agriculturally-acceptable carrier.

13. A peptide composition for the inhibition of fungal pathogen growth, the peptide composition comprising a nucleotide structure FHWWXPSYSVPS, where X can be any amino acid and Y can be any amino acid.

14. The peptide composition of claim 13, where at least one of X and Y is alanine.

15. The peptide composition of claim 13, further comprising an agriculturally-acceptable carrier.

16. The peptide composition of claim 13, further comprising a pharmaceutically acceptable carrier.

17. A method of treating an agricultural product to inhibit fungal pathogen growth, comprising:
introducing the peptide composition of claim 13 to at least one of soil configured to support an agricultural product or a seed as a seed coating.

18. The method of claim 17, wherein the peptide composition includes an agriculturally-acceptable carrier.


SEQUENCE LISTING (41 sequences)

SEQ ID NO: 1 - protein, 12 aa, synthetic construct
KVWTIPNGALGS

SEQ ID NO: 2 - protein, 12 aa, synthetic construct
YSTTGHVTYVGR

SEQ ID NO: 3 - protein, 12 aa, synthetic construct
AGTGVPMRLGGL

SEQ ID NO: 4 - protein, 12 aa, synthetic construct
IPLVDTDYVTSR

SEQ ID NO: 5 - protein, 12 aa, synthetic construct
VIAKHTRPVMAG

SEQ ID NO: 6 - protein, 12 aa, synthetic construct
AHGHTPSLTDSM

SEQ ID NO: 7 - protein, 12 aa, synthetic construct
GYRHVPMSPPMG

SEQ ID NO: 8 - protein, 12 aa, synthetic construct
GHKHDNLSMESL

SEQ ID NO: 9 - protein, 12 aa, synthetic construct
GHKHTFEITPRA

SEQ ID NO: 10 - protein, 12 aa, synthetic construct
SHVHVHGGYKQK

SEQ ID NO: 11 - protein, 12 aa, synthetic construct
GHKHVSLPPWVE

SEQ ID NO: 12 - protein, 12 aa, synthetic construct
GHKHIQYPRYNA

SEQ ID NO: 13 - protein, 12 aa, synthetic construct
LPVSHPHRMQTD

SEQ ID NO: 14 - protein, 12 aa, synthetic construct
IVTVGQPLTGPK

SEQ ID NO: 15 - protein, 12 aa, synthetic construct
WHWWPWVDSQNT

SEQ ID NO: 16 - protein, 12 aa, synthetic construct
HLPTWVMWPLSA

SEQ ID NO: 17 - protein, 12 aa, synthetic construct
WHWTWFSQAMMA

SEQ ID NO: 18 - protein, 12 aa, synthetic construct
FHWWWPSPSVPS

SEQ ID NO: 19 - protein, 12 aa, synthetic construct
GHWSIRMTTYPP

SEQ ID NO: 20 - protein, 12 aa, synthetic construct
WHWWWPSPSLPS

SEQ ID NO: 21 - protein, 12 aa, synthetic construct
WHWWWPSPSVPS

SEQ ID NO: 22 - protein, 12 aa, synthetic construct
WHWWWPSPSLGS

SEQ ID NO: 23 - protein, 12 aa, synthetic construct
WLWWWPSRSLGS

SEQ ID NO: 24 - protein, 12 aa, synthetic construct
AHWWWPSPSVPS

SEQ ID NO: 25 - protein, 12 aa, synthetic construct
FAWWWPSPSVPS

SEQ ID NO: 26 - protein, 12 aa, synthetic construct
FHAWWPSPSVPS

SEQ ID NO: 27 - protein, 12 aa, synthetic construct
FHWAWPSPSVPS

SEQ ID NO: 28 - protein, 12 aa, synthetic construct
FHWWAPSPSVPS

SEQ ID NO: 29 - protein, 12 aa, synthetic construct
FHWWWASPSVPS

SEQ ID NO: 30 - protein, 12 aa, synthetic construct
FHWWWPAPSVPS

SEQ ID NO: 31 - protein, 12 aa, synthetic construct
FHWWWPSASVPS

SEQ ID NO: 32 - protein, 12 aa, synthetic construct
FHWWWPSPAVPS

SEQ ID NO: 33 - protein, 12 aa, synthetic construct
FHWWWPSPSAPS

SEQ ID NO: 34 - protein, 12 aa, synthetic construct
FHWWWPSPSVAS

SEQ ID NO: 35 - protein, 12 aa, synthetic construct
FHWWWPSPSVPA

SEQ ID NO: 36 - protein, 15 aa, synthetic construct
MAACPIGETFDKTLR

SEQ ID NO: 37 - protein, 15 aa, synthetic construct
FRGRAALSDQFKDYR

SEQ ID NO: 38 - protein, 12 aa, synthetic construct
GHKHTSLPPWVA

SEQ ID NO: 39 - protein, 12 aa, synthetic construct
  SITE 1: note = D-Serine
  SITE 2: note = D-Histidine
  SITE 3: note = D-Tryptophan
  SITE 4: note = D-Tryptophan
  SITE 5: note = D-Tryptophan
  SITE 6: note = D-Histidine
  SITE 7: note = D-Serine
  SITE 8: note = D-Glycine
  SITE 9: note = D-Serine
  SITE 10: note = D-Lysine
  SITE 11: note = D-Serine
  SITE 12: note = D-Serine
SHWWWHSGSKSS

SEQ ID NO: 40 - protein, 12 aa, synthetic construct
  VARIANT 5: note = X can be any amino acid
  VARIANT 8: note = X can be any amino acid
FHWWXPSXSVPS

SEQ ID NO: 41 - protein, 12 aa, synthetic construct
SHWWWHSGSKSS